(19) United States
(12) Patent Application Publication
(10) Pub. No.: US 2006/0183978 A1
(43) Pub. Date: Aug. 17, 2006

(54) Laryngoscope

(76) Inventor: Gregory L. Howard, Sugar Land, TX (US)

Correspondence Address: Loren G. Helmreich;Browning Bushman P.C., Suite 1800, 5718 Westheimer, Houston, TX, 77057, US

(21) Appl. No.: 11/057,644

(22) Filed: Feb. 14, 2005

(51) Int. Cl. A61B 1/267

(52) U.S. Cl. 600/197; 600/190; 600/199

(57) ABSTRACT

A laryngoscope 10 includes an elongate blade 20 for insertion into the patient, a handle 12 for manipulating the blade, and a connecting member 30 interconnecting the blade and the handle. The handle axis 14 is laterally spaced from a plane of a blade axis 22 such that the handle is exterior of the patient's mouth when manipulating the blade. In one embodiment, the connecting member 30 has a connecting member axis 34 angled at about 90° relative to the plane of the blade axis 22. The laryngoscope blade is removably connected to the handle, and is fixably secured to the blade.

### FIELD OF THE INVENTION

The present invention relates to a laryngoscope suitable for tracheal intubation of patients. The laryngoscope may be used to visualize the laryngeal area of the body, and significantly reduces the likelihood of damage to the patient's teeth.

### BACKGROUND OF THE INVENTION

Laryngoscopes are commonly used to facilitate endotracheal intubation during surgery to permit the patient to breathe and/or to administer anaesthesia. The patient's head is conventionally tilted backwards as far as possible and the lower jaw distended to open the mouth widely. A rigid blade, which may be straight or curved, may be inserted through the mouth into the throat passageway to expose the glottis.

There are many types of laryngoscope blades. Many have a channel along the blade to help guide the endotracheal tube during insertion into the larynx. Some blades have complex contours and shapes. The laryngoscope blade is preferably rigid, durable and sterilizable. To achieve these goals, laryngoscope blades have been made of metal, including stainless steel or chrome plated brass.

Intubation of a trachea protects the patient's airway during general anaesthesia and may be used to ventilate the patient using positive pressure. A common practice during intubation is to cease ventilating the patient, insert the laryngoscope, visualize the opening of the trachea and insert an endotracheal tube. Laryngoscopes are often used by physicians and anesthetists in patient operating rooms, and are used by emergency workers at accident scenes and in emergency transport vehicles.

Conventional laryngoscopes frequently contact the upper teeth so that the blade or handle is pivoted about the edge of these teeth during use with the patient's teeth acting as a fulcrum. This practice results when the handle is grasped to force the blade against the tongue and throat of a patient to expand and open the mouth and throat for viewing and intubation. A light source is commonly used to illuminate the throat area of the patient.

A common complaint of the laryngoscope involves damage to the teeth of the patient. The force applied by the laryngoscope is often sufficient to chip or break the patient's teeth. Not only may there be cosmetic disfigurement, but discomfort and extensive restoration dentistry. If a patient aspirates a dislodged tooth or fragment, there may be pulmonary complications. Some prior art devices have a connecting member between the molars, but the handle configuration does not allow for force to be applied to the molars during difficult intubation.

Various techniques have been proposed to minimize damage to the teeth caused by use of the laryngoscope, including modified laryngoscopes, teeth protectors and laryngoscope pads. Relevant technology includes U.S. Pat. Nos. 4,384,570; 4,425,909; 4,437,458; 4,546,762; 4,565,187; 4,570,614; 4,574,784; 4,579,108; 4,583,527; 4,592,343; 4,611,579; 4,799,485; 4,827,910; 4,905,669; 4,947,896; 5,003,963; 5,033,480; 5,063,907; 5,065,738; 5,070,859; 5,178,132; 5,363,840; 5,438,976; 5,498,231; 5,536,245; 5,776,053; 5,827,178; 5,879,304; 6,095,972; 6,174,281; 6,217,514; 6,257,236; 6,471,643; 6,494,828; 6,623,425; 6,626,829; 6,666,819; 6,676,598; and 6,764,443.

The disadvantages of the prior art are overcome by the present invention, and an improved laryngoscope is hereinafter disclosed.

### SUMMARY OF THE INVENTION

In one embodiment, a laryngoscope is used with a conventional handle for manipulating a laryngoscope blade. The handle may be a generally cylindrical member having a handle axis. The elongate laryngoscope blade is manipulated by the handle for insertion into the patient, and includes a curved or a straight blade axis lying within the blade axis plane. A connecting member, which may be in the form of a rigid shaft, interconnects the blade and the handle. The handle axis is laterally spaced from the blade axis plane by a spacing of at least two inches, such that handle is exterior of the patient's mouth when manipulating the blade. The connecting member may be fixedly secured to the blade and removably connected to the handle. The connecting member has an axis angled at least 45°, and in one embodiment about 90°, relative to the blade axis plane. A lighting line, which may be the formal fiber optic line, extends from the handle, along with connecting member, and along at a portion of the elongate blade for illuminating an area adjacent the blade.

### BRIEF DESCRIPTION OF THE DRAWINGS

FIG. 1 illustrates one embodiment of an laryngoscope including an elongate blade, a handle and a connecting member connecting the blade and the handle.

FIG. 2 illustrates the laryngoscope handle shown in FIG. 1 structurally removed from the connecting member and the blade.

FIG. 3 illustrates the laryngoscope blade positioned within the mouth of a patient and the handle laterally external of the patient's mouth.

### DETAILED DESCRIPTION OF THE EMBODIMENTS

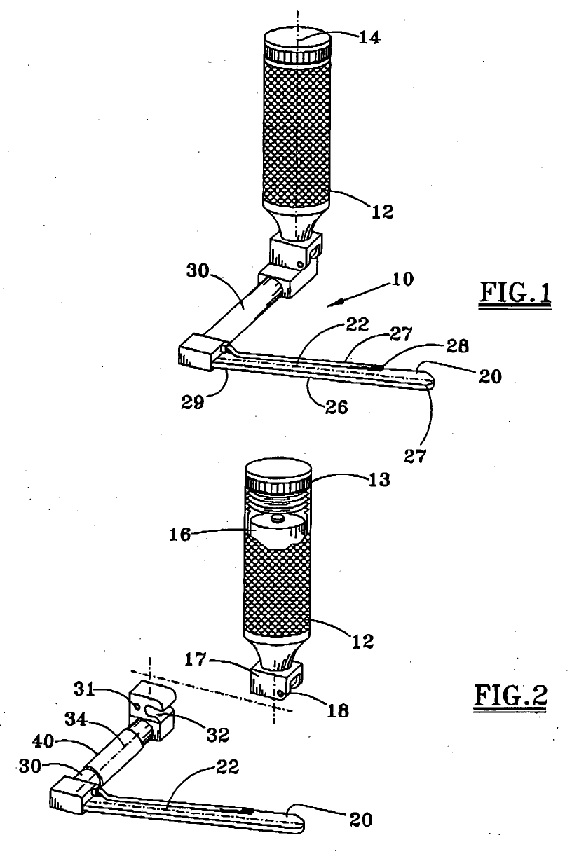

Referring to FIG. 1, the laryngoscope 10 includes a conventional handle 12 having a generally cylindrical configuration for manipulating the laryngoscope blade 20. The handle 12 includes a handle axis 14, and conventionally includes a battery or other power source 16 (see FIG. 2) for illuminating an area adjacent the blade. An end cap 13 may be removed from the body of the handle to obtain access to the interior of the handle.

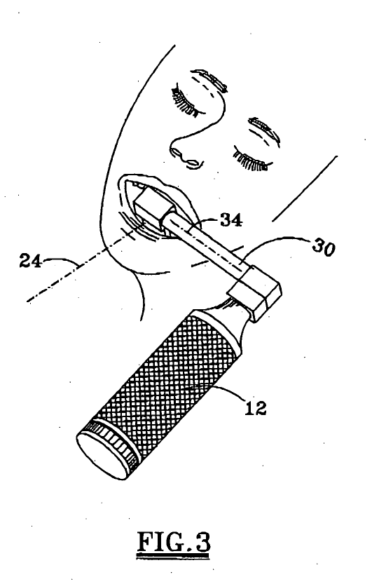

The elongate blade 20 is configured for insertion into the patient, and may have a relatively straight axis or a curved axis. In either event, the elongate blade axis 22 lies within a blade axis plane 24 (see FIG. 3) which is in use generally centered with respect to the mouth and throat of the patient. The configuration of the blade will depend on the desires of the practitioner and the physical characteristics of the patient. A substantially straight blade is shown in FIG. 1, although a blade with a curved axis may be used. The blade 20 generally has a smooth bottom surface 26 running from its free end 27 toward the attached end 29.

The blade 20 is designed for attachment to a handle 12. Any suitable attachment means for releasably mounting the blade 20 to the handle 12 may be provided. An end section 17 of the handle 12 is typically provided with opposed spring biased balls or detents for releasably locking engagement with complimentary shaped balls or detent recesses in the handle, as shown in FIG. 2. The pin 18 on the handle 12 allows the blade to pivot between a locked position and a disengaged position. An electrical connection can also be used to provide electrical current from batteries 16 carried in the handle 12 for powering a light 28 on the blade 20 via lighting line 27.

Connecting member 30 interconnects the blade 20 and the handle 12, and positions the handle axis laterally from the blade axis plane by spacing of at least two inches, such that the handle 12 is exterior of the patient's mouth when manipulating the blade, as shown in FIG. 3. Connecting member 30 may be fixedly secured to the blade 20, and may be removably connected to the handle 12 by a conventional latching device, which may comprise a slot 32 for positioning the pin 18 therein, and balls or detents 31 for cooperation with similarly configured balls or detents in the handle to lock the connecting member 30 to the handle in a locked position. The connecting member 30 in the blade 20 may be removed from the handle 12 by pivoting the connecting member 30 with respect to the handle 12 to disengage the balls and detents, and then moving the connecting members so that the slot 32 disengages from the pin 18. The connecting member has a connecting member axis 34 angled at least 45° with respect to the blade axis 22, and as shown in FIG. 1, the connecting member axis is angled at about 90° relative to the blade axis plane. In one embodiment, the connecting member laterally spaces the handle axis from the blade axis plane by at least two inches, and in some embodiments at least three inches. The end of the handle 12 is removably connected to a side of the connecting member 30, which may comprise a rigid shaft.

It is preferable if axis 14 of the handle is positioned less than 90° and greater than 45° with respect to axis 22 of the blade 20. This will allow the distal end of the blade 20 to be positioned above horizontal during direct laryngoscopy of an anterior airway while axis 14 of the handle is still directed anterior with respect to the patient. This will help avoid the natural tendency to use the molars as a fulcrum. In alternate embodiments, the axis 14 of the handle could be spaced from the axis 34 of the connecting member 30, and may be spaced closer to the tip of the blade compared to axis 34. In still other embodiments, axis 14 may be spaced slightly rearward of the tip of the blade with respect to axis 34. The blade and the connecting member are preferably formed from stainless steel or chrome plated brass.

A lighting line 27 extends from the handle, along the connecting member, and along at least a portion of the elongate blade for illuminating an area adjacent the blade. The lighting line may be a fiber optic line, or may be an electrical line for powering a miniature bulb.

During use, the practitioner may select the desired configuration of a blade 20 with a connecting member 30 fixed thereon, then interconnect the blade and connecting member 30 to the handle 12, as discussed above. The blade is then inserted into the throat of the patient, and the connecting member 30 will extend laterally outward from the mouth of the patient. If the connecting member 30 is angled upward at, e.g, 30° from the embodiment shown in FIG. 3 so that the handle end of the connecting member 30 is higher than the blade 30 is higher than the blade end of the connecting member, (connecting member axis angled at 60° relative to plane 24) the blade will be inserted slightly farther into the mouth of the patient than if the connecting member 30 is at 90° from the plane 24 which includes the axis 22 of the blade. In either event, the connecting member 30 will preferably engage the molars of the patient when the handle 12 is rotated to fully open the patient's airway. If desired, a plastic or other cushioning protective coating layer 40 on the connecting member may be provided to minimize abrasion to the molars.

Once the blade 20 is properly inserted, the practitioner may then rotate the handle from the position as shown in FIG. 3 upward, thereby moving the blade in a manner similar to prior art laryngoscopes which used a handle 12 in line with the plane 24 of the blade axis. A significant advantage to the laryngoscope disclosed herein is that the front teeth of the patient are not engaged by either the handle or the blade. The connecting member 30 which contacts the molars of the patient ideally exerts no appreciable force on the molars. However, during difficult intubation, there will be a tendency to use the teeth as a fulcrum. The molars can better be protected than the upper front teeth.

In an alternate embodiments, the blade may be removably connected to the blade end of a connecting member with a mechanism similar to the removable connection between the handle and the connecting member. In yet another embodiment, the blade and handle may each be fixably connected to the connecting member. A light source may be provided in the handle, and a fiber optic line provided on the blade for viewing the throat area.

Although specific embodiments of the invention have been described herein in some detail, this has been done solely for the purposes of explaining the various aspects of the invention, and is not intended to limit the scope of the invention as defined in the claims which follow. Those skilled in the art will understand that the embodiment shown and described is exemplary, and various other substitutions, alterations and modifications, including but not limited to those design alternatives specifically discussed herein, may be made in the practice of the invention without departing from its scope.

1. A laryngoscope, comprising:
an elongate blade for insertion into the patient, the elongate blade having a blade axis lying within a blade axis plane;
a handle for manipulating the blade, the handle having a handle axis; and
a connecting member interconnecting the blade and the handle, such that the handle axis is laterally spaced from the blade axis plane, such that the handle is exterior of the patient's mouth when manipulating the blade.

2. A laryngoscope as defined in claim 1, wherein the connecting member is fixably secured to the blade and is removably connected to the handle.

3. A laryngoscope as defined in claim 1, wherein the connecting member has a connecting member axis angled at least 45° with respect to the blade axis plane.

4. A laryngoscope as defined in claim 1, wherein the connecting member has a connecting member axis angled at about 90° relative to the blade axis plane.

5. A laryngoscope as defined in claim 1, further comprising:
a lighting line extending from the handle, along with connecting member, and along at least a portion of the elongate blade for illuminating an area adjacent the blade.

6. A laryngoscope as defined in claim 5, wherein the lighting line is a fiber optic line.

7. A laryngoscope as defined in claim 1, wherein the connecting member laterally spaces the handle axis from the blade axis plane by at least two inches.

8. A laryngoscope as defined in claim 1, wherein the handle is removably connected to a side of the connecting member.

9. A laryngoscope as defined in claim 1, wherein the connecting member is a rigid shaft.

10. A laryngoscope blade for use with a handle for manipulating the blade, the handle having a handle axis, comprising:
the blade configured for insertion into the patient, the blade having a blade axis lying within a blade axis plane;
a connecting member interconnecting the blade and the handle, such that the handle axis is laterally spaced from the blade axis plane by a spacing of at least two inches, such that the handle is exterior of the patient's mouth when manipulating the blade;
the connecting member is fixably secured to the blade and is removably connected to the handle; and
the connecting member has a connecting member axis angled at least 45° with respect to the blade axis plane.

11. A laryngoscope blade as defined in claim 10, wherein the connecting member has a connecting member axis angled at about 90° relative to the blade axis plane.

12. A laryngoscope blade as defined in claim 10, further comprising:
a lighting line extending from the handle, along with connecting member, and along at least a portion of the elongate blade for illuminating an area adjacent the blade.

13. A laryngoscope blade as defined in claim 10, wherein the connecting member laterally spaces the handle axis from the blade axis plane by at least two inches.

14. A laryngoscope blade as defined in claim 1, wherein the handle is removably connected to a side of the connecting member.

15. A laryngoscope blade for use with a handle for manipulating the elongate blade, the handle having a handle axis, comprising:
the elongate blade configured for insertion into the patient, the elongate blade having a blade axis lying within a blade axis plane;
a connecting member interconnecting the blade and the handle, such that the handle axis is laterally spaced from the blade axis plane by a spacing of at least two inches, such that the handle is exterior of the patient's mouth when manipulating the blade; and
the connecting member has a connecting member axis angled at about 90° relative to the blade axis.

16. A laryngoscope blade as defined in claim 15, further comprising:
a lighting line extending from the handle, along with connecting member, and along at least a portion of the elongate blade for illuminating an area adjacent the blade.

17. A laryngoscope blade as defined in claim 16, wherein the lighting line is a fiber optic line.

18. A laryngoscope blade as defined in claim 15, wherein the connecting member is fixably secured to the blade and removably connected to the handle.

19. A laryngoscope blade as defined in claim 15, wherein the handle is removably connected to a side of the connecting member.

20. A laryngoscope blade as defined in claim 15, wherein the connecting member is a rigid shaft.
